(19) United States
(12) Patent Application Publication
(10) Pub. No.: US 2008/0119669 A1
(43) Pub. Date: May 22, 2008

(54) Process for the Recovery of Organic Compounds from Mixtures Containing (Meth)Acrylic Acid by Extraction with a Protic Solvent

(76) Inventors: Torsten Balduf, Pfungstadt (DE); Andreas Sabbagh, Dulmen (DE); Thomas Nehrkorn, Marl (DE); Manfred Lucas, Schermbeck (DE)

Correspondence Address: SMITH MOORE LLP, P.O. BOX 21927, GREENSBORO, NC, 27420, US

(21) Appl. No.: 11/815,633

(22) PCT Filed: Feb. 7, 2006
(86) PCT No.: PCT/EP06/01077

§ 371 Date: Sep. 14, 2007

(30) Foreign Application Priority Data
Feb. 8, 2005 (DE) 10 2005 005 439.0

(51) Int. Cl. C07C 51/42; B01J 8/00

(52) U.S. Cl. 562/600; 422/187

(57) ABSTRACT

The invention relates the preparation of (meth)acrylic acid comprising the steps: i) bringing a composition Z₁ comprising (meth)acrylic acid and aldehydes into contact with an aldehyde-trapping agent to give a composition Z₂ comprising (meth)acrylic acid, the high-boiling reaction product of the aldehyde, and the aldehyde-trapping agent, and unreacted aldehyde-trapping agent; ii) separating off (meth)acrylic acid from the composition Z₂ by means of distillation, whereby a composition Z₃ comprising the high-boiling reaction product from the reaction between the aldehyde and the aldehyde-trapping agent and unreacted aldehyde-trapping agent is being obtained as the bottom product; iii) extracting the unreacted aldehyde-trapping agent from the composition Z₃ by water, a first, aqueous phase P₁ and a second, organic phase P₂ being obtained; iv) separating off of the first, aqueous phase P₁ from the second, organic phase P₂.

This application is a national stage application under 35 U.S.C. 371 of international application No. PCT/EP2006/001077 filed 7 Feb. 2006, and claims priority to German Application No. DE 10 2005 005 439.0 filed 8 Feb. 2005, the disclosures of which are expressly incorporated herein by reference.

### BACKGROUND OF THE INVENTION

The present invention relates to a process for the preparation of (meth)acrylic acid, a device for the preparation of (meth)acrylic acid, foams, shaped articles, fibers and the like based on (meth)acrylic acid obtained by the process according to the invention and the use of the (meth)acrylic acid obtained by the process according to the invention in such products.

In the present context, (meth)acrylic acid is understood as meaning both methacrylic acid and acrylic acid, acrylic acid being preferred.

(Meth)acrylic acid, and in particular acrylic acid, is a monomer which is used in many polymers. In particular, acrylic acid is used in the preparation of polymers which are employed for water treatment, for example as flocculating agents, or may be incorporated as superabsorbent polymers into hygiene articles, in particular diapers (see. Modern Superabsorbent Polymer Technology, F. L. Buchholz, A. T. Graham; Wiley-VCH 1998).

It is furthermore generally known that acrylic acid often and methacrylic acid also frequently can be prepared by heterogeneous catalyzed gas phase oxidation of propylene or isobutene with oxygen on catalysts which are in general in the solid state of aggregation, at temperatures of between 200 and 400° C. Reference is made in this connection to DE OS 19 62 431, DE OS 29 43 707 and to DE 108 38 845 A1.

It is furthermore known from WO 03/051809 A1 to bring a product gas comprising (meth)acrylic acid obtained from the gas phase oxidation of propylene into contact with an aqueous phase to give an aqueous quenched phase. In such a quenched phase, the (meth)acrylic acid is still accompanied by various other reaction products, which are regarded as impurities, and moreover by water as an absorption agent. This fact generally makes it necessary to feed the quenched phase obtained above to at least one further purification process. This purification process is often a distillation in which, in particular, the absorption agent is separated off, optionally in the presence of an entraining agent, and a so-called crude (meth)acrylic acid is obtained.

This crude (meth)acrylic acid can then be further purified by distillation for the purpose of separating off low- or high-boiling by-products still present.

Generally, the crude (meth)acrylic acid or the crude (meth)acrylic acid which has optionally been purified further by distillation still contains an amount of aldehydes which is not to be ignored, such as, for example, benzaldehyde or furfural. However, aldehydes impede the polymerization of the acrylic acid and moreover lead to discolored polymers. Furthermore, these aldehydes are unacceptable for health reasons, so that (meth)acrylic acid, which is employed in particular for the production of hygiene articles, must have a particularly high purity in respect of aldehydes.

WO 03/014172 A1 therefore proposes addition of so-called aldehyde-trapping agents which react with the aldehydes to form high-boiling reaction products to the crude (meth)acrylic acid. The (meth)acrylic acid can then be separated of from the crude (meth)acrylic acid comprising these high-boiling reaction products by distillation. The bottom product comprising the high-boiling reaction products that are obtained in this distillation is generally disposed of by combustion.

The aldehyde-trapping agents are usually employed in excess with respect to the aldehydes contained in the crude (meth)acrylic acid, in order to ensure as complete as possible a conversion of the aldehydes into the high-boiling reaction products. The consequence of this, however, is that unreacted aldehyde-trapping agent contained in the bottom product is lost during the disposal referred to above for the bottom product, which is a disadvantage in particular for reasons of cost. Furthermore, in the cases where mercaptans are employed as aldehyde-trapping agents, the high contents of sulfur compounds in the bottom product leads to a significant pollution of the environment during combustion thereof.

In general, the present invention was based on the object of overcoming the disadvantages arising from the prior art.

### SUMMARY

An embodiment of the present invention is directed to a process for the preparation of (meth)acrylic acid, comprising the following process steps: i) bringing a composition Z₁ comprising (meth)acrylic acid and aldehydes into contact with an aldehyde-trapping agent at a temperature in a range of from about 10° C. to about 100° C. under a pressure in a range of from about 0.1 to about 10 bar to give a composition Z₂ comprising Z₂a (meth)acrylic acid; Z₂b a reaction product of the aldehyde and the aldehyde-trapping agent; and Z₂c unreacted aldehyde-trapping agent; ii) separating off at least some of (meth)acrylic acid from the composition Z₂ to give a composition Z₃ comprising, Z₃a the reaction product from the reaction between the aldehyde and the aldehyde-trapping agent; and Z₃b unreacted aldehyde-trapping agent; iii) extracting the unreacted aldehyde-trapping agent from the composition Z₃ by a protic solvents, a first, more protic phase P₁ and a second phase P₂ which is less protic compared with the phase P₁ being obtained; and iv) separating off of the first phase P₁ from the second phase P₂.

### FIGURE

The foregoing and other features, aspects, and advantages of the present invention will become better understood with regard to the following description, appended claims, and accompanying drawing where:

FIG. 1 is a schematic of an embodiment of the process according to the present invention.

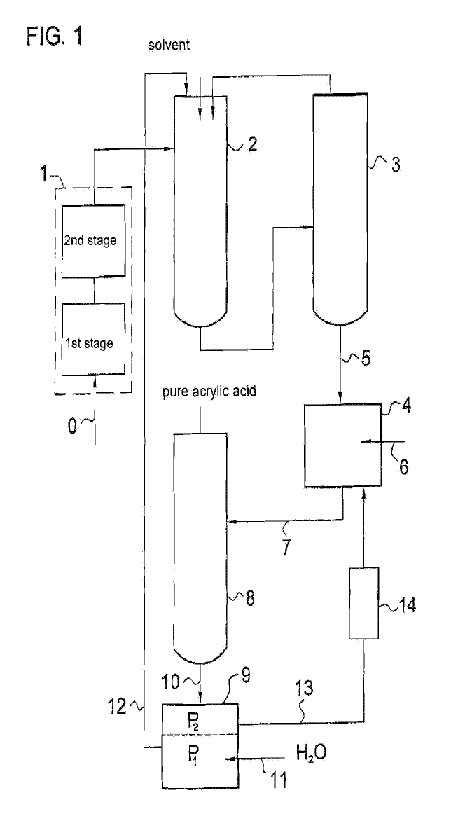

### DESCRIPTION OF THE PRESENT INVENTION

In particular, the present invention was based on the object of providing a process with which (meth)acrylic acid can be prepared with an extremely low residual content of aldehydes, and therefore with the smallest possible toxicological problems, as inexpensively as possible.

This process should furthermore render possible a preparation of (meth)acrylic acid that is as environment-friendly as possible.

The present invention was also based on the object of providing a device with which this process can be operated.

A contribution towards achieving the abovementioned objects is provided by a process for the preparation of (meth)acrylic acid comprising the following process steps:

i) bringing of a composition Z₁ comprising (meth)acrylic acid and aldehydes into contact with an aldehyde-trapping agent at a temperature in a range of from 10 to 100° C. under a pressure in a range of from about 0.1 to about 10 bar to give a composition Z₂ comprising
Z₂a (meth)acrylic acid,
Z₂b a preferably high-boiling reaction product of the aldehyde and the aldehyde-trapping agent and
Z₂c unreacted aldehyde-trapping agent;

ii) separating off at least some of (meth)acrylic acid from the composition Z₂ to give a composition Z₃ comprising
Z₃a the preferably high-boiling reaction product from the reaction between the aldehyde and the aldehyde-trapping agent and
Z₃b unreacted aldehyde-trapping agent;

iii) extracting the unreacted aldehyde-trapping agent from the composition Z₃ by a protic solvents, preferably with an aqueous phase, particularly preferably with water, wherein a first, more protic, preferably aqueous phase P₁ and a second, preferably organic phase P₂ which is less protic compared with the phase P₁ is being obtained;

iv) separating off of the first phase P₁ from the second phase P₂.

It has been found, surprisingly, but no less advantageously, that unreacted aldehyde-trapping agent can be separated off from compositions containing (meth)acrylic acid in a simple manner by extraction with a solvent, preferably with water. In this context, at least 50 wt. % of unreacted aldehyde-trapping agent can be recovered.

“High-boiling” compounds in the context of the present invention are understood as meaning compounds that have a boiling point under atmospheric pressure of higher than 160° C. in the case of the preparation of acrylic acid, and of higher than 180° C. in the case of the preparation of methacrylic acid. Conversely, “readily boiling” or “low-boiling” compounds in the context of the present invention are understood as meaning compounds which have a boiling point under atmospheric pressure of less than 120° C. in the case of the preparation of acrylic acid and of less than 140° C. in the case of the preparation of methacrylic acid.

### Process Step I

In a preferred embodiment of the process according to the invention, the composition Z₁ employed in process step i) is a composition which has been obtained by a process comprising the process steps:

a) catalytic gas phase oxidation of C₃-C₄-hydrocarbons with oxygen to give a product gas mixture containing (meth)acrylic acid, the product gas mixture containing aldehydes as by-products;

b) absorption of the product gas mixture in a solvent and subsequent separating off of the solvent to give the composition Z₁

or
fractional condensation of the product gas mixture and separating off of the composition Z₁. 5/28

The oxidation of the C₃-C₄-hydrocarbons, which are preferably propane, propylene, and/or acrolein in the case of the preparation of acrylic acid, and preferably isobutylene in the case of the preparation of methacrylic acid, in the gas phase in process step a) to give (meth)acrylic acid is carried out in a manner known. The feed, which is optionally mixed with an inert dilution gas, is passed in a mixture with oxygen at elevated temperatures, conventionally from about 200 to about 400° C., and optionally under increased pressure over at least one heterogeneous catalyst, as a rule transition metal mixed oxide catalysts containing e.g. molybdenum, vanadium, tungsten, and/or iron, and is thereby converted oxidatively into (meth)acrylic acid. The reaction can be carried out in one stage or two stages. In the case of the preparation of (meth)acrylic acid, in a two-stage reaction procedure the propylene or isobutylene preferably employed as the starting compound is oxidized to (meth)acrolein in a first stage and the (meth)acrolein is oxidized to (meth)acrylic acid in a second stage. Preferred heterogeneous catalysts are oxidic multi-component catalysts based on the oxides of molybdenum, bismuth and iron in the first stage and corresponding catalysts based on the oxides of molybdenum and vanadium in the second stage.

The reaction of propane, propylene, or isobutylene to give (meth)acrylic acid is highly exothermic. The feed stream is therefore advantageously diluted with an inert dilution gas, e.g. atmospheric nitrogen, carbon dioxide, methane, and/or water vapor. Although the nature of the reactors used is not subject to any limitation per se, tubular bundle heat exchangers which are filled with the oxidation catalyst(s) are expediently used, since in these the predominant portion of the heat released during the reaction can be removed by convection and radiation at the cooled tube walls. In addition to (meth)acrylic acid, the reaction gases obtained during the one- or two-stage catalytic gas phase oxidation conventionally contain unreacted starting compounds, water vapor, carbon monoxide, carbon dioxide, nitrogen, oxygen, acetic acid, propionic acid, formaldehyde, further aldehydes, and maleic acid, or maleic anhydride.

In process stage b), the working up of the product gas mixture obtained in process step a) now starts. In this context, two different procedures are conceivable.

In one embodiment of the process according to the invention, (meth)acrylic acid is absorbed from the reaction gases in absorption liquid. Liquids in which (meth)acrylic acid has a pronounced solubility, e.g. liquids which boil at a higher temperature than (meth)acrylic acid, are suitable as the absorption liquid. Diphenyl, diphenyl ether, dimethyl phthalate, ethylhexanoic acid, N-methylpyrrolidone, paraffin fractions, or mixtures thereof are suitable e.g. as the high-boiling liquid. Alternatively, mixtures containing oligomeric acrylic acids, such as di-, tri- and tetraacrylic acid, can be employed as the high-boiling liquid. Diphenyl, diphenyl ether, o-dimethyl phthalate, or mixtures thereof are preferred, in particular a mixture of from about 25 to about 30 wt. % diphenyl and from about 70 wt % to about 75 wt % diphenyl ether which contains from about 0.1 wt % to about 25 wt % o-dimethyl phthalate, based on the mixture.

In a particularly preferred embodiment of the process according to the invention, water is employed as the absorption liquid.

The absorption liquid is brought intimately into contact with the product gas mixture in a suitable manner. For this, the product gas mixture is expediently led in an absorption column in countercurrent to the descending absorption liquid. A filled, packed, valve tray or bubble tray column e.g. can be employed as the absorption column.

The reaction gases, which as a rule have a temperature of from about 200° C. to about 400° C., are preferably cooled to a suitable absorption temperature of, for example, about 100° C. to about 180° C. before introduction into the absorption column. The cooling of the reaction gases to the absorption temperature can be carried out by indirect cooling. e.g. by means of a heat exchanger. Preferably, however, this cooling is carried out by direct contact with a cooling liquid, preferably in a spray washer. The cooling liquid is expediently largely separated off again in a separator, cooled and recycled before entry of the reaction gases into the absorption column. The cooling liquid is preferably identical to the liquid used for subsequent absorption of the acrylic acid from the reaction gases.

In addition to (meth)acrylic acid, the absorption liquid loaded with (meth)acrylic acid as a rule still contains volatile impurities, such as water, acrolein, formaldehyde, formic acid, and acetic acid. Secondary components, such as water, acrolein, formaldehyde, and acetic and formic acid, can be at least partly removed by stripping with a stripping gas, especially if a high-boiling liquid is employed as the absorption liquid. For this, the absorption liquid loaded with (meth)acrylic acid is fed in a desorption column in countercurrent to a stripping gas, such as e.g. nitrogen or air. The amount of stripping gas required depends above all on the desorption temperature, which is advantageously chosen from about 20° C. to about 50° C. higher than the absorption temperature. The procedure is preferably carried out under the same pressure as in the absorption step. The amount of stripping gas is preferably, based on the amount of reaction gas, from about 5 vol % to about 25 vol %. The desorption column can be e.g. a filled, packed, valve tray or bubble tray column.

The cooling liquid and/or the absorption liquid conventionally contain e.g. an amount of from about 0.01 wt % to about 1 wt % of at least one process polymerization inhibitor, such as phenothiazine, phenolic compounds, such as hydroquinone, hydroquinone monomethyl ether, p-nitrosophenol, tert-butylphenols, 1-oxyl-2,2,6,6-tetramethylpiperidin-4-ol, or mixtures thereof

The composition Z₃, also called crude (meth)acrylic acid, is then isolated from the absorption liquid loaded with (meth)acrylic acid. If a high-boiling liquid is employed as the absorption liquid, the crude (meth)acrylic acid is conventionally separated off by rectification. The separating off by rectification is expediently carried out under reduced pressure, e.g. 0.04 to 0.1 bar, e.g. in a filled or tray column. A polymerization inhibitor is advantageously added at the top or in the upper region of the rectification column. In this context, the crude (meth)acrylic acid can be removed as the top product; preferably, however, it is removed by a side take-off in the upper region of the rectification column, small amounts of impurities which have lower boiling points than (meth)acrylic acid, such as water and acetic acid, being taken off at the top of the column. The high-boiling liquid obtained after the crude (meth)acrylic acid has been separated off is expediently recycled and used again for the absorption. Under certain circumstances, it is advantageous to subject all or some of the residue consisting chiefly of the high-boiling liquid to heat treatment at temperatures above about 180° C. before it is recycled into the absorption column, whereby ester-like oligomeric (meth)acrylic acids present as an impurity being cleaved and the (meth)acrylic acid formed being distilled off together with the high-boiling liquid. The maleic acid still present, especially in the case of preparation of acrylic acid, or its anhydride can be removed in a conventional manner per se, e.g. by extraction with water, before re-use of the high-boiling liquid.

If, in the manner preferred according to the invention, water is used as the absorption liquid for absorption of the (meth)acrylic acid from the reaction gases, the crude (meth)acrylic acid is expediently isolated by extraction with and extraction agent and subsequent distillation of the extract from the aqueous (meth)acrylic acid solution primarily obtained. The extraction agent should have a high partition coefficient for (meth)acrylic acid and a low solubility in water, and it must form an azeotrope with water. Extraction agents which have lower boiling points than (meth)acrylic acid, such as ethyl acetate, butyl acetate, ethyl acrylate, 2-butanone or mixtures thereof, or extraction agents which have higher boiling points than (meth)acrylic acid may be used. Toluene is a preferred extraction agent in the preparation of acrylic acid.

For the extraction, the aqueous (meth)acrylic acid solution is suitably led in an extraction column in countercurrent to the extraction agent chosen.

Crude (meth)acrylic acid is then separated off from the extract by distillation. The distillation procedure depends on whether an extraction agent having a higher or lower boiling point than (meth)acrylic acid is used. In the case of a use, which is particularly preferred according to the invention, of an extraction agent of lower boiling point than (meth)acrylic acid, the extract is fed, for example, to a solvent separation column in which the extraction agent and residual amounts of water are distilled off over the top. The bottom fraction of the solvent separation column is then advantageously fed to a low-boiling substances column, in which impurities of lower boiling point than (meth)acrylic acid are separated off over the top and crude (meth)acrylic acid is obtained as the bottom fraction. A further separating off of high-boiling impurities from this crude (meth)acrylic acid by a further distillation step is also conceivable.

Instead of isolating the composition Z₁ from the reaction gases by absorption in an absorption liquid, crude (meth)acrylic acid can also be obtained by fractional condensation of the reaction gases, optionally with subsequent purification by crystallization.

For the fractional condensation, the reaction gases, the temperature of which has preferably been reduced to e.g. from about 100° C. to about 180° C. by direct cooling with a cooling liquid, are expediently passed into the lower region of a column with incorporated units having a separating action, and are allowed to ascend within the column. A crude (meth)acrylic acid fraction can be removed as composition Z₁ as a medium-boiling fraction via a suitably attached collecting tray. Such a process is described in DE 197 40 253 and DE 197 40 252. As a rule a process polymerization inhibitor, such as those mentioned above, is introduced into the column.

The crude (meth)acrylic acid fraction of composition Z₁ obtained during the fractional condensation can be fed to a crystallization for the purpose of further purification. The crystallization process is not subject to any limitation. The purification by crystallization, if used, is advantageously carried out as a suspension crystallization.

Regardless of the nature of the working up of the product gas mixture, a crude (meth)acrylic acid comprising aldehydes is obtained as composition Z₁.

In a particularly preferred embodiment of the process according to the invention, a crude (meth)acrylic acid which has been obtained as the bottom product by absorption of the product gas mixture with water in a quenching tower and subsequent separating off of the water by azeotropic distillation in the presence of toluene as an entraining agent is employed as composition Z₁, low- and high-boiling impurities still present advantageously having been separated off by further distillation steps.

In another embodiment of the invention, the composition Z₁ may be based on
from about 95 wt % to about 99.99 wt %, or from about 98 wt % to about 99.98 wt %, or from about 99 wt % to about 99.97 wt. % (meth)acrylic acid,
from about 1 to about 2,000 ppm, or from about 1 to about 1,000 ppm, or from about 1 to about 500 ppm aldehydes, which are, for example, benzaldehyde, acrolein, or furfural in the case of the preparation of acrylic acid,
from about 0.001 wt % to about 1 wt %, or from about 0.01 to about 0.5 wt %, or from about 0.05 wt % to about 0.2 wt % water,
up to about 1 wt %, or up to about 0.5 wt %, or up to about 0.2 wt. % of dimeric or oligomeric (meth)acrylic acid, and
up to about 1 wt %, or up to about 0.5 wt %, or up to about 0.1 wt % of further impurities.

In process step i), the composition Z₁ is brought into contact with an aldehyde-trapping agent at a temperature in a range of from about 10° C. to about 100° C., or in a range of from about 10° C. to about 70° C., or in a range of from about 20° C. to about 30° C., room temperature being most preferred, under a pressure in a range of from about 0.1 bar to about 10 bar, or in a range of from about 0.5 bar to about 5 bar, or in a range of from about 0.9 bar to about 2 bar, atmospheric pressure, to give a composition Z₂ comprising (meth)acrylic acid, the high-boiling reaction product from the aldehyde, and the aldehyde-trapping agent, and unreacted aldehyde-trapping agent.

Aldehyde-trapping agents which can be employed according to the invention are all compounds which form preferably high-boiling reaction products with aldehydes under the abovementioned pressure and temperature conditions.

Possible aldehyde-trapping agents which may be mentioned are nitrogen compounds with at least one primary amino group, such as, for example, aminoguanidine salts, hydrazine, alkyl- and arylhydrazines, carboxylic acid hydrazides, or aminophenols. In an embodiment of the process which is particularly preferred according to the invention, however, mercaptans, preferably C₆- to C₂₀-mercaptans, particularly preferably C₈- to C₁₆-mercaptans are used. The mercaptans which are most preferred in this connection include dodecylmercaptan.

The aldehyde-trapping agent is preferably employed in excess with respect to the aldehyde contained in the crude (meth)acrylic acid, preferably in an amount of from about 1.1 mol to 5 mol, or from about 1.5 to about 2.5 mol per mol of aldehyde. A reaction time of from about 10 minutes to about 72 hours, or from about 1 hour to about 50 hours, or from about 1.1 to about 10 hours is conventionally maintained. By the treatment with the aldehyde-trapping agent, the residual aldehyde content of the crude (meth)acrylic acid can be lowered to below about 20 ppm, or below about 5 ppm, or below about 3 ppm.

The bringing of the composition Z₁ into contact with the aldehyde-trapping agent can be carried out, for example, by introducing the aldehyde-trapping agent directly into a pipeline by means of which the crude (meth)acrylic acid is fed to the further working up. It is also conceivable to add the aldehyde-trapping agent into a dwell tank in which the crude (meth)acrylic acid is stored intermediately, before it is fed to the further working up.

In another embodiment of the process according to the invention, the composition Z₁ is brought into contact with the aldehyde-trapping agent in a fixed bed reactor. This fixed bed reactor may be a reactor comprising a reaction chamber and a stationary packed bed (a porous fixed bed) in the reaction chamber. The porous fixed bed in this context is preferably introduced in a loose packing on a support, such as, for example, a filter, arranged in the reactor. The porous fixed bed may comprise bulk filling bodies, such as Raschig rings, Berl saddles, Intalox saddles, or Pall rings, or spherical filling bodies, spherical filling bodies being most preferred. In another embodiment, the filling bodies may be based on an ion exchanger material, such as a zeolite material. According to another embodiment of the process according to the invention, a fixed bed reactor may comprise ion exchanger beads assembled in a loose packing on a sieve is used.

### Process Step II

In process step ii), the (meth)acrylic acid is at least partly separated off from the composition Z₂ obtained in process step i), which, in addition to the (meth)acrylic acid, comprises the preferably high-boiling reaction products from the reaction between the aldehyde-trapping agent and the aldehyde and unreacted aldehyde-trapping agent. The composition Z₂ accordingly substantially differs from the composition Z₁ in that it comprises less (meth)acrylic acid compared with the composition Z₁. In this context, an embodiment my include more than about 50 wt %, or more than about 75 wt %, or more than about 95 wt %, or more than about 99 wt % of the (meth)acrylic acid contained in the composition Z₁ to be separated off.

In another embodiment of the process according to the invention, this separating off is carried out by distillation. The term “separating off by distillation” is intended to include in this context both a simple distillation, i.e. a distillation in which substantially no exchange of matter takes place between the condensate and vapors, and a rectification, in which some of the condensate is led in countercurrent to the ascending vapors. The distillation in process step ii) can be carried out by means of distillation devices known to the person skilled in the art.

In the distillation in process step ii), on the one hand a pure (meth)acrylic acid may be obtained as the target product as the top product or in a side stream (depending on the nature of the distillation process chosen), while a composition Z₃ which, in addition to the (meth)acrylic acid still present, comprises the preferably high-boiling reaction product from the aldehyde and the aldehyde-trapping agent and unreacted aldehyde-trapping agent may be obtained as the bottom product.

In another embodiment of the process according to the invention, this composition Z₃ is based on:
from about 10 wt % to about 99 wt %, or from about 40 wt % to about 99 wt %, or from about 50 wt % to about 99 wt % of (meth)acrylic acid,
up to about 2 wt %, or up to about 1 wt %, or up to about 0.5 wt % of high-boiling reaction products from the reaction between the aldehyde and the aldehyde-trapping agent,
from about 0.1 wt % to about 10 wt %, or from about 0.5 wt % to about 5 wt %, or from about 1 wt % to about 2 wt % of unreacted aldehyde-trapping agent,
from about 1 wt % to about 30 wt %, or from about 2 wt % to about 20 wt. %, or from about 5 to about 10 wt % of dimeric or oligomeric (meth)acrylic acid, and
from about 0.5 wt % to about 15 wt %, or from about 0.5 wt % to about 10 wt %, or from about 1 wt % to about 5 wt. % of other impurities.

### Process Steps III) And IV

In process step iii), the unreacted aldehyde-trapping agent may be extracted by means of a protic solvent, or by means of an aqueous phase, or by means of water, from the bottom product (with composition Z₃) obtained in process step ii).

In this context the composition Z₃ may be brought into contact with from about 1 wt % to about 75 wt %, or from about 2.5 wt % to about 50 wt %, or from about 5 wt % to about 25 wt % of the solvent, or the aqueous phase, or water, in each case based on the weight of the composition Z₃, the bringing into contact being carried out at a temperature in a range of from about 15° C. to 50° C., or from about 20° C. to about 30° C., under an absolute pressure in a range of from about 0.5 bar to about 5 bar, or from about 0.9 bar to about 2 bar.

In the extraction of the composition Z₃ with the solvent, with the water, a first, more protic, phase P₁ and a second, organic phase P₂ which may be less protic compared with the phase P₁ are obtained. In this context the aldehyde-trapping agent may become concentrated in one of the two phases P₁ or P₂. Become more concentrated means of more than about 50 wt %, or more than about 60 wt %, or more than about 70 wt % or more than about 90 wt % of the amount of unreacted aldehyde-trapping agent contained in the composition Z₃ before the extraction with the solvent is in the first phase P₁ or the second phase P₂, or in the second phase P₂, after the extraction. If water is used as the solvent and mercaptans as the aldehyde-trapping agent, the majority of the unreacted mercaptan is in the second, organic phase P₂ after the extraction.

The first aqueous phase P₁ obtained in the context of the extraction is preferably based on
from about 30 wt % to about 95 wt %, or from about 40 to about 90 w. %, or from about 50 wt % to about 85 wt % (meth)acrylic acid,
from about 5 wt % to about 40 wt %, or from about 5 wt % to about 30 wt %, or from about 15 wt % to about 25 wt % water,
up to about 5 wt. %, or up to about 2 wt %, or up to about 1 wt % of aldehyde-trapping agent,
from about 0.5 wt % to about 10 wt %, or from about 1 wt % to about 5 wt %, or from about 1 wt % to about 3 wt % of dimeric or oligomeric (meth)acrylic acid, and
from about 0.5 wt % to about 10 wt %, from about 1 wt % to about 5 wt %, or from about 1 wt % to about 2 wt % of further impurities.

The second, preferably organic phase P₂ obtained in the context of the extraction is preferably based on
from about 15 wt % to about 60 wt %, or from about 20 wt % to about 50 wt %, or from about 25 wt % to about 45 wt % (meth)acrylic acid,
from about 0.01 wt to about 10 wt %, or from about 0.05 wt % to about 5 wt %, or from about 0.1 wt % to about 2 wt % water,
from about 30 wt % to about 95 wt %, or from about 40 wt % to about 90 wt %, or from about 50 wt % to about 85 wt % of aldehyde-trapping agent,
from 0 to about 5 wt %, or from about 0.1 wt % to about 2 wt %, or from about 0.1 wt % to about 1 wt. % of dimeric or oligomeric (meth)acrylic acid, and
from about 0.1 wt % to about 10 wt %, or from about 0.2 wt % to about 5 wt %, or from about 1 wt % to about 2 wt % of further impurities.

In process step iv), the first, an aqueous phase P₁ is separated off from the second, an organic phase P₂.

The extraction of the composition Z₃ obtained in process step ii) with water in process step iii) and the subsequent separation of the two phases obtained in this manner in process step iv) may be achieved by any device which renders possible an extraction and a subsequent separation of the phases obtained in the course of the extraction.

An “extraction” as used herein means any process with which a compound from a starting phase (the composition Z₃) in which it is dissolved or suspended, becomes concentrated in another liquid phase (second organic phase P₂). In the case of a discontinuous operating procedure, extraction by shaking is also referred to, and in a continuous procedure perforation.

The separating off of the first, an aqueous phase P₁ from the second, an organic phase P₂ may be carried out in a procedure known to the person skilled in the art in connection with conventional extraction processes. The separating off can be carried out in a particularly simple manner with suitable devices for separating off, such as, for example, a separating funnel. In respect of the general procedure in the extraction, one skilled in the art may make reference to Thornton J. D., “Science and Practice of Liquid-Liquid Extraction”, vol. I & II, Oxford, Oxford University Press, 1992, to Lo T.-C., Baird M. H. I and Hanson C., “Handbook of Solvent Extraction”, New York, John Wiley & Sons, 1983 and to Robbins G. M. and Cusack R. W., “Liquid-Liquid Extraction Operations and Equipment” in “Perry's Chemical Engineering Handbook”, Perry R. H. and Green D. W., New York, McGraw Hill, chapter 15, 1997.

The extraction in process step iii) may be operated in a multi-stage extraction process on a large industrial scale to achieve high extraction yields with the simultaneous use of small amounts of water, for example by means of a mixer-separator combination, or with a mixer-separator cascade, or by means of the use of extraction columns.

In another embodiment, the second, an organic phase P₂ obtained after the separating off in process step iv) is recycled, optionally after a separating off of impurities, such as, for example, high-boiling substances, oligomers or polymers, for example by means of filtration or distillation, into process step i) in a further process step v) following process step iv). By this method and manner, unreacted aldehyde-trapping agent is available for further conversion of the aldehydes into high-boiling compounds. The first, an aqueous phase P₁ obtained after the separating off in process step iv) may be recycled, for example, into process step b) of the process for the preparation of crude (meth)acrylic acid, in particular into process step b) in which an absorption of the product gas mixture in water is carried out.

A further contribution towards achieving the abovementioned objects is provided by a device for the preparation of (meth)acrylic acid comprising, as device constituents connected to one another by fluid-carrying lines:

(δ1) a (meth)acrylic acid reactor;

(δ2) a quenching device connected to the (meth)acrylic acid reactor (δ1)

or
a condensation device connected to the (meth)acrylic acid reactor (δ1);

(δ3) optionally one or more distillation devices for separating off low- and/or high-boiling substances connected to the quenching device (δ2)

or
a crystallization device connected to the condensation device (δ2);

(δ4) a reactor, preferably a fixed bed reactor, connected to the quenching device (δ2) or the distillation device (δ3) or the crystallization device (δ3), comprising:
(δ4_{—}1) a feed for a composition comprising aldehydes;
(δ4_{—}2) a feed line for an aldehyde-trapping agent;
(δ4_{—}3) a feed line for a recycled aldehyde-trapping agent;
(δ4_{—}3) a removal line for a composition comprising reaction products from the reaction between the aldehydes and the aldehyde-trapping agent and unreacted aldehyde-trapping agent;

(δ5) a further distillation device connected to the reactor (δ4);

(δ6) an extraction device connected to the bottom of the further distillation device (δ5), comprising
(δ6_{—}1) a feed line for a composition comprising reaction products from the reaction between the aldehydes and the aldehyde-trapping agent and unreacted aldehyde-trapping agent;
(δ6_{—}2) a feed line for a solvent;
(δ6_{—}3) a first removal line for a first, aqueous phase P₁;
(δ6_{—}4) a second removal line for a second, organic phase P₂;
   wherein the second removal line (δ6_{—}4) is connected to the reactor (δ4), and optionally the first removal line (δ6_{—}3) is connected to the quenching device (δ2) or to the condensation device (δ2).

According to the invention, fluid-carrying means that the lines, preferably pipelines, are constructed and configured such that these can lead gases or liquids or hypercritical fluids or solids suspended in liquid or at least two of these.

In another embodiment of the device, the reactor (δ5) is a fixed bed reactor, wherein this may be characterized by the features already mentioned in connection with the description of the process according to the invention.

The extraction device (δ6) is a device that may comprise both a mixing unit and a unit for separating off substances. These units for mixing and separating off substances may be individual device constituents connected by fluid-carrying lines, such as is the case, for example, in the preferred mixer-separator cascade. However, it is also conceivable for these two device units to be combined to a single device constituent, such as is the case, for example, with the extraction column.

In another embodiment, the first removal line (δ6_{—}3) is connected to the quenching device (δ2).

The present invention also relates to a process for the preparation of (meth)acrylic acid, in which the device described above may be employed.

The present invention furthermore relates to foams, shaped articles, fibers, foils, films, cables, sealing materials, superabsorbers, liquid-absorbing hygiene articles, carriers for plant and fungal growth-regulating compositions, packaging materials, soil additives or building materials based on (meth)acrylic acid obtained by the process according to the invention described above. “Based” in this context means that these are built up to the extent of at least about 10 wt %, or at least about 25 wt %, or at least about 50 wt. % on this (meth)acrylic acid.

The present invention furthermore relates to the use of (meth)acrylic acid obtained by the process according to the invention in foams, shaped articles, fibers, foils, films, cables, sealing materials, superabsorbers, liquid-absorbing hygiene articles, carriers for plant and fungal growth-regulating compositions, packaging materials, soil additives, for controlled release of active compounds, or in building materials.

The invention will now be explained in more detail with the aid of non-limiting FIGURE and example.

The FIGURE shows the process according to the invention and a device according to the invention for the preparation of (meth)acrylic acid, in which the product gas mixture is absorbed in water in a quenching tower and the water is then separated off in a distillation column.

The gaseous starting compounds (in the case of the preparation of acrylic acid a gas mixture of propene, oxygen, water vapor and nitrogen) are introduced via feed line 0 into the reactor 1 and are converted there, optionally in two reaction stages, into acrylic acid and further gaseous reaction products. The product gas mixture obtained in the reactor 1 is then transferred into a quenching tower 2, in which the acrylic acid and further by-products are absorbed in water to give an aqueous acrylic acid solution. The aqueous acrylic acid solution obtained in the bottom of the quenching tower is introduced into a distillation column, in which the water is removed by means of azeotropic distillation in the presence of toluene. The bottom product (crude acrylic acid) obtained in the distillation column 3 is introduced into a reactor 4, which is preferably a fixed bed reactor. It is also conceivable for the bottom product obtained in the distillation column 3 first also to be further purified by distillation and only then to be introduced into the reactor 4 (not shown). An aldehyde-trapping agent is introduced via feed line 6 into the reactor 4 and is brought into contact there with the bottom products, so that high-boiling reaction products are formed from the reaction between the aldehyde-trapping agent and the aldehydes still contained in the bottom product. The composition obtained in this way is transferred via the removal line 7 into a further distillation device 8, in which pure acrylic acid is distilled off at the top. The bottom product obtained in the further distillation column 8 is transferred via feed line 10 into an extraction device 9. In a particular embodiment of the process according to the invention, impurities, such as, for example, high-boiling substances, oligomers or polymers, can be removed by means of a separating device, which is, for example, a distillation or filtration device, from the bottom product obtained in this way before transfer into the extraction device 9 (not shown). Water as the extraction agent is introduced into the extraction device 9 via the feed line 11. After the extraction, two phases P₁ and P₂ are formed in the extraction device 9, the lighter, organic phase P₂, which comprises above all unreacted aldehyde-trapping agent, being recycled via the removal line 13, which is connected to the reactor 4, back into the reactor 4. In this context, impurities, such as, for example, high-boiling substances, oligomers or polymers, can be removed by means of a separating device 14, which is, for example, a distillation or filtration device, from the organic phase P₂ before the recycling into the reactor 4. It is particularly advantageous if impurities, particularly preferably solid-like impurities, are removed by means of a separating device, preferably by means of a filtration device as the separating device, both from the organic phase P₂ before the recycling into the reactor 4 and from the bottom product obtained in the distillation column 8, before the transfer into the extraction device 9. The first, aqueous phase P₁ obtained in the extraction device 9 can be recycled via the removal line 12 back into the quenching device 2.

### EXAMPLE

200 g of a bottom product, containing dodecylmercaptan as an aldehyde-trapping agent, of the column for separating off high-boiling substances are mixed with the amounts of water stated in the table at room temperature under atmospheric pressure in a 250 ml separating funnel. After intensive shaking over a period of 3 minutes, a settling time of from 8 to 10 hours followed, in order to separate the phases from one another. The two phases were then drained off and weighed. For the analysis, the samples were additionally centrifuged at 4,000 rpm in order to remove a slight clouding, and were then analyzed. At a water content of 9% in total in the total mixture, 9.9 g of upper phase and 209.6 g of lower phase resulted. This corresponded to a dodecylmercaptan recovery of 56%. At a water content of 17% in the total mixture, 15.5 g of upper phase and 223.9 g of lower phase were found. This corresponded to a dodecylmercaptan recovery of 85%.

The analysis is carried out via gas chromatography with a thermal conductivity detector (all figures in wt. %).

| | H₂O | AA¹⁾ | DMC²⁾ | PA³⁾ | MEHQ⁴⁾ | dAA⁵⁾ | PA⁶⁾ | SR⁷⁾ | MAA⁸⁾ |
|---|---|---|---|---|---|---|---|---|---|
| Educt | | 87.96 | 3.66 | 0.055 | 2.30 | 5.63 | 0.12 | 0.28 | 0.75 |
| (filtered) | | | | | | | | | |
| 9% H₂O | 9.29 | 84.35 | 1.88 | — | 1.37 | 3.01 | — | 0.10 | — |
| phase P₁ | | | | | | | | | |
| 9% H₂O | 0.57 | 58.38 | 38.93 | — | 0.45 | 1.25 | — | 0.12 | — |
| phase P₂ | | | | | | | | | |
| 17% H₂O | 10.40 | 77.00 | 0.48 | — | 1.29 | 2.66 | — | 0.17 | — |
| phase P₁ | | | | | | | | | |
| 17% H₂O | 0.46 | 41.88 | 56.44 | — | 0.32 | 0.67 | — | 0.23 | — |
| phase P₂ | | | | | | | | | |

¹⁾Acrylic acid
²⁾Dodecylmercaptan
³⁾Propionic acid
⁴⁾Methylhydroquinone
⁵⁾Dimeric acrylic acid
⁶⁾Protoanemonin
⁷⁾Residue which can be separated off
⁸⁾Malic acid/maleic anhydride

### LIST OF REFERENCE SYMBOLS

0 Feed for reaction gases

1 (Meth)acrylic acid reactor

2 Quenching tower

3 Distillation column

4 Reactor for conversion of aldehydes into high-boiling compounds

5 Feed for the bottom product from the distillation column 3 into the reactor 4

6 Feed for an aldehyde-trapping agent into the reactor 4

7 A removal line for a composition comprising high-boiling reaction products from the reaction between the aldehydes and the aldehyde-trapping agent and unreacted aldehyde-trapping agent, via which this composition can be transferred into a further distillation column 8

8 Further distillation column

9 Extraction device

10 Feed line for the bottom product from the distillation column 8 into the extraction device 9

11 Feed line for water

12 Removal line for a first aqueous phase

13 Removal line for a second organic phase, via which this phase can be recycled into the reactor 4.

14 Device for sluicing out impurities

1. A process for the preparation of (meth)acrylic acid, comprising the following process steps:
i) bringing a composition Z₁ comprising (meth)acrylic acid and aldehydes into contact with an aldehyde-trapping agent at a temperature in a range of from about 10 to about 100° C. under a pressure in a range of from about 0.1 to about 10 bar to give a composition Z₂ comprising
Z₂a (meth)acrylic acid,
Z₂b a reaction product of the aldehyde and the aldehyde-trapping agent and
Z₂c unreacted aldehyde-trapping agent;
ii) separating off at least some of (meth)acrylic acid from the composition Z₂ to give a composition Z₃ comprising
Z₃a the reaction product from the reaction between the aldehyde and the aldehyde-trapping agent and
Z₃b unreacted aldehyde-trapping agent;
iii) extracting the unreacted aldehyde-trapping agent from the composition Z₃ by a protic solvents, a first, more protic phase P₁ and a second phase P₂ which is less protic compared with the phase P₁ being obtained; and
iv) separating off of the first phase P₁ from the second phase P₂.

2. The process according to claim 1, wherein the composition Z₁ is obtained by a process comprising the process steps:
a) catalytic gas phase oxidation of C₃-C₄-hydrocarbons with oxygen to give a product gas mixture containing (meth)acrylic acid, the product gas mixture containing aldehydes as by-products;
b) absorption of the product gas mixture in a solvent and subsequent separating off of the solvent to give Z₁
or
fractional condensation of the product gas mixture and separating off of the composition Z₁.

3. The process according to claim 1, wherein the second phase P₂ obtained in process step iv) is recycled into process step i).

4. The process according to claim 1, wherein the aldehyde-trapping agent is a mercaptan compound.

5. The process according to claim 4, wherein the mercaptan compound is a C₆- to C₂₀.

6. The process according to claim 1, wherein in process step ii) the composition Z₃ is extracted with about 2.5 to about 50 wt. % of the solvent, based on the weight of the composition Z₃.

7. The process according to claim 1, wherein the extraction and the separating off in process steps iii) and iv) are carried out by means of at least one mixer-separator combination, preferably a mixer-separator cascade, or by means of an extraction column.

8. A device for the preparation of (meth)acrylic acid, comprising, as device constituents connected to one another by fluid-carrying lines:
(δ1) a (meth)acrylic acid reactor;
(δ2) a quenching device connected to the (meth)acrylic acid reactor;
or
a condensation device connected to the (meth)acrylic acid reactor;
(δ3) optionally one or more distillation devices for separating off low- and/or high-boiling substances connected to the quenching device;
or
a crystallization device connected to the condensation device;
(δ4) a reactor connected to the quenching device or the distillation device or the crystallization device, comprising:
(δ4_{—}1) a feed for a composition comprising aldehydes;
(δ4_{—}2) a feed line for an aldehyde-trapping agent;
(δ4_{—}3) a removal line for a composition comprising reaction products from the reaction between the aldehydes and the aldehyde-trapping agent and unreacted aldehyde-trapping agent;
(δ5) a further distillation device connected to the reactor;
(δ6) an extraction device connected to the further distillation device, comprising:
(δ6_{—}1) a feed line for a composition comprising reaction products from the reaction between the aldehydes and the aldehyde-trapping agent and unreacted aldehyde-trapping agent;
(δ6_{—}2) a feed line for a solvent;
(δ6_{—}3) a first removal line for a first phase P₁;
(δ6_{—}4) a second removal line for a second phase P₂;
wherein the second removal line is connected to the reactor.

9. A device according to claim 8, wherein the reactor is a fixed bed reactor.

10. A device according to claim 8, wherein the first removal line is connected to the quenching device.

11. The process according to claim 1 wherein a device according to claim 7 is used.

12. Foams, shaped articles, fibres, foils, films, cables, sealing materials, liquid-absorbing hygiene articles, carriers for plant and fungal growth-regulating compositions, packaging materials, soil additives or building materials based on (meth)acrylic acid obtained by a process according to claim 1.

13. Use of (meth)acrylic acid obtained by a process according to claim 1 to in foams, shaped articles, fibres, foils, films, cables, sealing materials, liquid-absorbing hygiene articles, carriers for plant and fungal growth-regulating compositions, packaging materials, soil additives, for controlled release of active compounds, or in building materials.
